(19) United States
(12) Patent Application Publication
(10) Pub. No.: US 2008/0193399 A1
(43) Pub. Date: Aug. 14, 2008

(54) COMPOSITION COMPRISING ARGAN OIL AND A PLANT-BASED PRODUCT OF THE ALOE GENUS, AND THE COSMETIC USE THEREOF

(76) Inventor: Philippe ALLEON, Inezgane (MA)

Correspondence Address: STITES & HARBISON PLLC, 1199 NORTH FAIRFAX STREET, SUITE 900, ALEXANDRIA, VA, 22314, US

(21) Appl. No.: 12/031,514

(22) Filed: Feb. 14, 2008

(30) Foreign Application Priority Data
Feb. 14, 2007 (MA) 29681
May 14, 2007 (FR) 07 55045

(51) Int. Cl. A61K 8/97; A61Q 5/00; A61Q 19/00; A61Q 19/08

(52) U.S. Cl. 424/61; 424/744; 424/74

(57) ABSTRACT

The invention relates to a composition comprising argan oil and an extract of Aloe; the use thereof for producing a cosmetic formulation and the resulting cosmetic formulation.

The invention relates to a composition for cosmetic use and the applications thereof.

The genus Aloe (division: Magnoliophyta, class: Liliopsida, order: Asparagales, family: Asphodelaceae) comprises approximately 400 species of succulent flowering plants, generally referred to as aloes, the most well known of which is undoubtedly Aloe vera. The majority of aloes have a cluster of wide, thick and spiked leaves in the form of a lance. They often have no stem, the cluster growing directly on the ground.

It is known to use products derived from Aloe vera in cosmetic preparations.

Argan oil is extracted from the fruits of the argan tree (Argania spinosa, sapotaceae family), argan seeds. This tree is little known since it grows only in the south-west region of Morocco over a surface-area of from 700,000-800,000 hectares.

The fruits of the argan tree are green. They resemble an olive but are larger and more round. Inside is a nut whose shell is very hard. This represents approximately one quarter of the weight of the fruit. The nut may contain up to three argan seeds, from which argan oil will be extracted by means of mechanical cold compression.

It is known that argan oil may be used in cosmetic preparations.

The patent application FR2553788 describes a method for preparing a lipidic extract of argan fruit.

The patent application FR2756183 describes a pharmaceutical or cosmetic composition which comprises a combination of argan oil and argan peptides.

The application WO 01/37792 describes dermatological compositions which comprise rice starch, coconut products, shea butter, borage oil, avocado oil, jojoba oil, and optionally 1.5% by weight of argan oil.

The patent application EP1764085 describes a cosmetic composition which comprises at least 10% by weight of argan oil.

Surprisingly, the Applicant has now discovered that the association of argan oil and a plant-based product of the Aloe genus allows a much higher level of activity to be obtained than that of argan oil alone or that of the plant-based product of the Aloe genus alone.

On the basis of this discovery, an object of the invention is to provide a composition which is intended for cosmetic use.

A second object of the invention is to provide cosmetic formulations which are intended for topical use.

In this manner, according to a first aspect, the present invention relates to a composition comprising argan oil and a plant-based product of the Aloe genus.

In the context of the present invention, the term “plant-based product of the Aloe genus” is intended to refer to a product which is derived from a plant of the Aloe genus, for example, a juice, a pulp, an extract, an oil, a lyophilisate, a zeodration product, a dehydration product or a mixture thereof obtained from an organ, a liquid or a ground material originating directly from a plant of the Aloe genus. Preferably, this derived product is obtained from the leaves.

In the context of the present invention, the term “plant of the Aloe genus” is preferably intended to refer to Aloe vera or Aloe arborescens and, in a particularly preferred manner, Aloe arborescens.

Advantageously, argan oil is present in the composition according to the invention at a ratio of from 10% to 40%, preferably from 20% to 30%, in particular 25% by weight of the composition.

Also in an advantageous manner, the plant-based product of the Aloe genus is present at a ratio of from 10% to 40%, preferably from 20% to 30%, in particular 25% by weight of the composition.

It should be emphasised that it is specifically the combined use of argan oil and a plant-based product of the Aloe genus which leads to the effect observed with the composition according to the invention. This combination has a significantly greater effect than that of argan oil alone or the same plant-based product of the Aloe genus alone, in particular a nourishing effect in the case of topical application to hair, skin or nails.

The compositions according to the invention may comprise, in addition to argan oil, one or more other vegetable oils, preferably selected from the group which is constituted by olive oil, avocado oil, shea oil, jojoba oil.

The compositions according to the invention may comprise, as an additional component, an extract of noni (Morinda citrifolia).

A method for preparing a composition according to the invention is an additional aspect of the present invention.

In a preparation method of this type, argan oil and a plant-based product of the Aloe genus are mixed in order to obtain an emulsion.

Another aspect of the present invention is the use of compositions according to the invention in order to prepare cosmetic formulations. Such cosmetic formulations also form part of the invention.

In order to ensure an optimal effect within the cosmetic formulation into which it is incorporated, the composition according to the invention is generally used in the formulation at a ratio of from 0.1 to 100% by weight, preferably at a ratio of from 30% to 40% by weight, in a particularly preferred manner at a ratio of from 40% to 50% by weight.

The cosmetic formulations according to the invention may comprise, in addition to the compositions according to the invention, one or more carriers which are acceptable in cosmetic terms.

Preferably, the cosmetic formulations according to the invention may comprise, as carriers which are acceptable in cosmetic terms, water, an organic solvent which can be mixed in water, natural oils, natural fatty acids or natural waxes, or mixtures thereof.

The term “carrier which is acceptable in cosmetic terms” is intended to refer to a carrier which is suitable for use in contact with human and animal cells, in particular the cells of the epidermis, without toxicity, irritation, undue allergic reaction and the like and which is proportioned at a reasonable benefit/risk ratio.

The cosmetic formulations according to the invention may comprise, in addition to the compositions according to the invention, one or more other additives which are acceptable in cosmetic terms and which are preferably selected from the group which is constituted by excipients, emulsifiers, surfactants, preservatives, perfumes, colourings, pigments and stabilisers.

Such cosmetic formulations according to the invention are generally in the form of a lotion, liquid emulsion, cream, milk, mousse, gel, aerosol or patch.

The person skilled in the art is capable of determining the type of the additional component(s) to use in the formulations of the invention taking into account the type of cosmetic formulation he wishes to obtain.

Another aspect of the invention is the use of a cosmetic formulation according to the invention as a topical application for nourishing, revitalising and/or firming the skin.

Another aspect of the invention is the use of a cosmetic formulation according to the invention as a topical application for nourishing and/or revitalising the hair.

Another aspect of the invention is the use of a cosmetic formulation according to the invention as a topical application for nourishing and/or revitalising the nails.

Another aspect of the invention is the use of a cosmetic formulation according to the invention as a topical application for the skin with an anti-ageing and/or anti-wrinkle activity.

Another aspect of the invention is the use of a cosmetic formulation according to the invention as a topical application for the skin for reducing age spots.

Preferably, the cosmetic formulation according to the invention is used as a topical application for the face and/or hands.

The following examples illustrate the invention, without limiting the scope thereof.

### EXAMPLE 1

A cosmetic formulation according to the invention is prepared in accordance with the following composition:

| | |
|---|---|
| ARGAN OIL | 40% |
| ALOES ARBORESCENS | 40% |
| SPERMACETI | 18% |
| HYDROGEN PEROXIDE | 1% |
| SODIUM BORATE | 0.5% |
| PERFUME EXTRACT | 0.5% |

This formulation is applied to the skin.

After a week of daily application, it is possible to observe:

softened and rehydrated skin,

a reduction of wrinkles,

a reduction of age spots.

1. Composition comprising argan oil and a plant-based product of the Aloe genus.

2. Composition according to claim 1, characterised in that the plant-based product of the Aloe genus is a leaf juice of a plant of the Aloe genus.

3. Composition according to claim 1, characterised in that the plant of the Aloe genus is Aloe arborescens.

4. Composition according to claim 1, characterised in that the argan oil is present at a ratio of from 20% to 30% by weight of the composition.

5. Composition according to claim 1, characterised in that the plant-based product of the Aloe genus is present at a ratio of from 20% to 30% by weight of the composition.

6. Cosmetic formulation comprising a composition according to claim 1.

7. Cosmetic formulation according to claim 6, characterised in that the formulation is a lotion, a liquid emulsion, a cream, a milk, a mousse, a gel or a patch.

8. Method for nourishing, revitalising and/or firming the skin, the hair and/or the nails, comprising a topical application of an acceptable amount of a cosmetic formulation according to claim 6.

9. Method for reducing wrinkles, comprising a topical application of an acceptable amount of a cosmetic formulation according to claim 6.

10. Method for reducing age spots, comprising a topical application of an acceptable amount of a cosmetic formulation according to claim 6.
